(19) United States
(12) Patent Application Publication
(10) Pub. No.: US 2022/0397460 A1
(43) Pub. Date: Dec. 15, 2022

(54) Wearable Environmental Sensor Device

(71) Applicant: Nippon Telegraph and Telephone Corporation, Tokyo (JP)

(72) Inventors: Akio Tokura, Tokyo (JP); Kei Kuwabara, Tokyo (JP); Takako Ishihara, Tokyo (JP); Hiroyoshi Togo, Tokyo (JP)

(21) Appl. No.: 17/783,523

(22) PCT Filed: Dec. 19, 2019
(86) PCT No.: PCT/JP2019/049953

§ 371 Date: Jun. 8, 2022

(51) Int. Cl. G01K 1/08; G01K 13/02

(52) U.S. Cl. CPC G01K 1/08; G01K 13/02; A41D 1/002

(57) ABSTRACT

A wearable environmental sensor is configured to measure environmental information regarding a place where the device is worn, and includes a black-bulb temperature sensor including a black bulb and a temperature sensor for measuring internal temperature in the black bulb, the black-bulb temperature sensor being in a housing, wherein the black bulb includes an insertion hole into which the temperature sensor is inserted, the black bulb includes a weld portion welded to the housing, in an outer-circumferential portion of the bottom surface, the black bulb includes a guide portion in an outer-circumferential portion around the insertion hole, the housing includes an insertion opening into which the guide portion of the black bulb is inserted, the housing includes a protruding portion at an outer-circumferential portion around the insertion opening, and the guide portion is supported by the protruding portion.

### CROSS-REFERENCE TO RELATED APPLICATIONS

This application is a national phase entry of PCT Application No. PCT/JP2019/049953, filed on Dec. 19, 2019, which application is hereby incorporated herein by reference

### TECHNICAL FIELD

The present invention relates to a wearable environmental sensor device for measuring environmental information regarding in the vicinity of a location where the device is worn, and in particular to a wearable environmental sensor device that is worn on clothing, a human body, or the like to measure information regarding the environment inside clothing of a wearer.

### BACKGROUND

It is important to monitor environmental information for physical condition management, such as prevention of heat stroke in hot weather. For example, heat index meters conventionally used to prevent heat stroke measure black-bulb temperature, wet-bulb temperature, and dry-bulb temperature to obtain a heat index, and a method is used in which the heat index is used as a guide of behavior, e.g., avoiding going out or doing strenuous work when the heat index is relatively high (see NPL 1).

A conventional heat index meter generally consists of a relatively large device, and it is difficult to place the device in any given location. For example, the heat index released by the Ministry of the Environment is a value that represents a wide area.

However, the actual heat load received by each individual is greatly influenced by the local environment. For example, the environment varies greatly depending on where each person is, such as outdoors or indoors, in the sun or in the shade, on the grass or on the concrete. In addition, even in the same location, the influence of radiation from the ground, for example, is greatly different between a tall adult and a short child. Moreover, the environment of the human body also varies greatly depending on the clothing worn, the state of movement, the state of perspiration, and so on.

### CITATION LIST

### Non Patent Literature

[NPL 1]—JuYoun Kwon, Ken Parsons, Evaluation of the Wet Bulb Globe Temperature (WBGT) Index for Digital Fashion Application in Outdoor Environments, Journal_Ergon_Soc_Korea 36 (2017) 23-36.

### SUMMARY

### Technical Problem

A method of carrying and wearing an environmental sensor is conceivable to monitor the environment in a desired place, particularly in the vicinity of a human body, but conventional WBGT meters or environmental sensors are too large in size to wear and inconvenient to carry, and there may be restrictions on the location where the sensor is worn.

Moreover, a small wearable environmental sensor device has not been known that can be attached onto individual's clothing or to the inside thereof and measure the environment in the immediate vicinity of the individual's clothing or the environment within the clothing.

Moreover, there is a problem that as the environmental sensor becomes smaller, it becomes more difficult to realize highly reliable formation and thermal insulation of the internal structure such as a black bulb.

An object of embodiments of the present invention is to measure the environment in the vicinity of a human body or the like accurately, easily, and stably.

### Means for Solving the Problem

To solve the foregoing problems, a wearable environment sensor device according to embodiments of the present invention is a wearable environment sensor device configured to measure environmental information regarding a place where the device is worn, the device including a black-bulb temperature sensor including a black bulb and a temperature sensor for measuring internal temperature in the black bulb, the black-bulb temperature sensor being provided in a housing, wherein the black bulb includes an insertion hole into which the temperature sensor is inserted, the insertion hole being provided in a bottom surface of the black bulb, the black bulb includes a weld portion welded to the housing, in an outer-circumferential portion of the bottom surface, the black bulb includes a guide portion in an outer-circumferential portion around the insertion hole, the housing includes an insertion opening into which the guide portion of the black bulb is inserted, the housing includes a protruding portion at an outer-circumferential portion around the insertion opening, and the guide portion is supported by the protruding portion, and the weld portion of the black bulb is welded to the housing in a vicinity of the outer-circumferential portion around the insertion opening of the housing.

### Effects of Embodiments of the Invention

With the wearable environmental sensor device according to the present, temperature, humidity, and environmental information can be measured accurately, easily, and stably.

### BRIEF DESCRIPTION OF THE DRAWINGS

FIG. 1A is an external view of front and back faces of a wearable environmental sensor device according to a first embodiment of the present invention.

FIG. 1B is an external view of side faces of the wearable environmental sensor device according to the first embodiment of the present invention.

FIG. 1C is a top view of the wearable environmental sensor device according to the first embodiment of the present invention.

FIG. 2 is a diagram showing examples of wearing the wearable environment sensor device according to the first embodiment of the present invention.

FIG. 3A is a diagram showing a housing surface before a black bulb is joined thereto in the wearable environment sensor device according to the first embodiment of the present invention.

FIG. 3B is a side view of a black bulb 1031 of the wearable environment sensor device according to the first embodiment of the present invention.

FIG. 3C is a cross-sectional view of the black bulb before a temperature sensor is arranged in the wearable environment sensor device according to the first embodiment of the present invention.

FIG. 3D is a cross-sectional view of the black bulb after the temperature sensor has been arranged in the wearable environment sensor device according to the first embodiment of the present invention.

FIG. 4 is a diagram showing protruding portions of the housing according to variations of the wearable environment sensor device according to the first embodiment.

FIG. 5 is a cross-sectional view of a joint portion between a black-bulb temperature sensor and a housing in a wearable environment sensor device according to a second embodiment.

### DETAILED DESCRIPTION OF ILLUSTRATIVE EMBODIMENTS

### First Embodiment

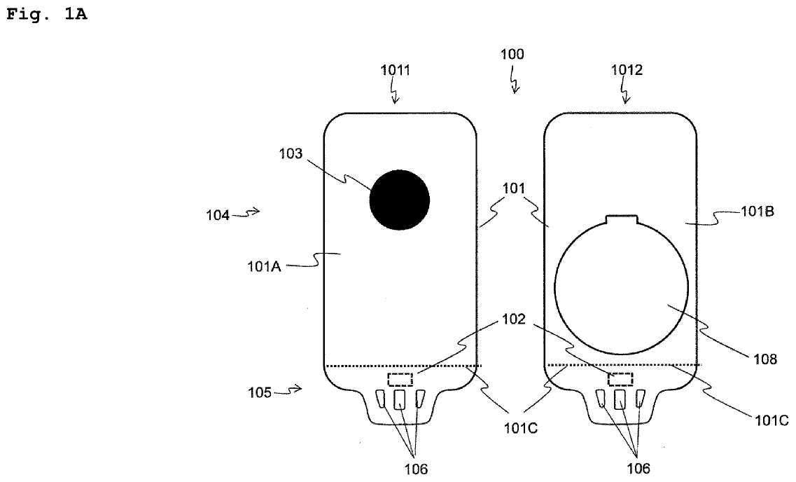

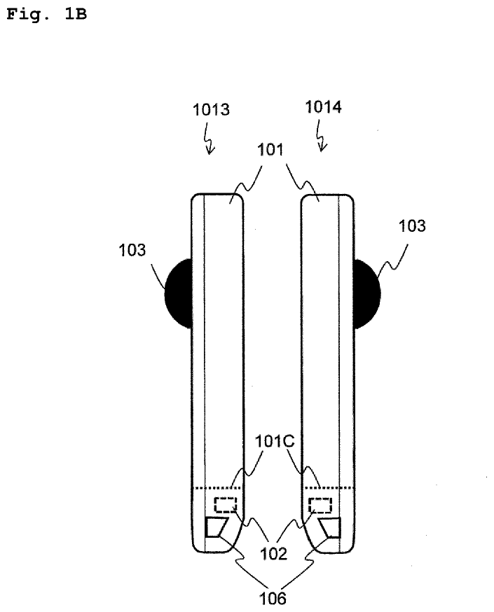

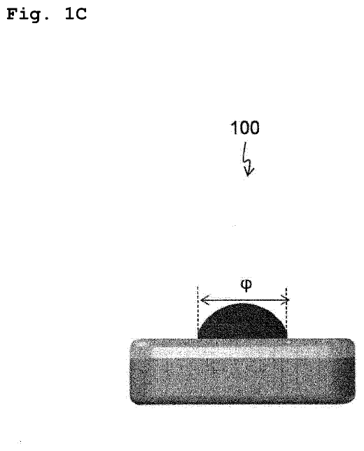

Hereinafter, a wearable environmental sensor device (hereinafter referred to as an “environmental sensor device”) 100 according to the first embodiment of the present invention will be described with reference to FIGS. 1 to 5.

FIG. 1 shows external views of the environmental sensor device 100 according to the present embodiment. FIG. 1A is an external view of a front face 1011 and a back face 1012 of the environmental sensor device 100. FIG. 1B is an external view of a right side face 1013 and a left side face 1014. FIG. 1C is a top view.

The environmental sensor device 100 has, in a housing 101, a temperature and humidity sensor 102 capable of measuring air temperature and relative humidity, and has, on a front face 101A of the housing 101, a black-bulb temperature sensor 103 for measuring black-bulb temperature. A lid 108 for battery replacement is provided in a back face 101B of the housing 101.

The environmental sensor device 100 includes a sealed portion 104 and a protective structure (unsealed portion) 105. The sealed portion 104 is sealed, and the sealed portion 104 and the protective structure (unsealed portion) 105 are separated by a housing wall 101C within the housing 101.

The size of the environmental sensor device 100 is about 50 mm in length, about 24 mm in width, and about 7 mm in thickness.

The temperature and humidity sensor 102 is mounted on a substrate (hereinafter referred to as “sensor substrate”), and the surface of the temperature and humidity sensor 102 on the side on which the temperature and humidity sensor 102 is mounted on the sensor substrate is referred to as a “sensor surface”. The temperature and humidity sensor 102 is arranged within the protective structure 105, and is covered by the protective structure for protecting the temperature and humidity sensor 102. The protective structure has a function of preventing the temperature and humidity sensor 102 from colliding with an external object and being damaged, and preventing a human finger or the like from coming into contact with the surface (sensor surface) of the temperature and humidity sensor 102 and making the sensor surface (sensor surface) dirty.

Further, since the temperature and humidity sensor 102 is arranged at an appropriate position with respect to the opening portions of the ventilating holes 106 provided in the protective structure (unsealed portion) 105, a structure is formed that makes it possible to sense changes in temperature and humidity due to ventilation and that is not exposed to water droplets coming from sweat or the like. Accordingly, it is possible to suppress a degradation of measurement accuracy of the temperature and humidity sensor 102 due to water droplets being attached to the sensor surface, and adverse effects such as a humidity value being observed higher than the actual humidity value.

It is desirable that the housing 101 is lightweight since it is wearable. It is also desirable that the housing 101 has low heat conduction such that heat is not transferred between the black-bulb temperature sensor 103, the housing 101, and the temperature and humidity sensor 102. For this reason, it is desirable that the material of the black bulb 1031 and the housing 101 is synthetic resin such as plastic, except for a region where metal needs to be used.

Only the battery lid 108 for inserting a button battery is provided in the back face of the housing 101 of the environmental sensor device 100 according to the present embodiment, and the environmental sensor device 100 can be worn by putting it into a pocket of clothing while operating the environmental sensor device 100 using a commercially available button battery.

As another mode, for example, a snap button, a clip, or the like may be provided on the back face of the housing 101 to attach the environmental sensor device 100 to clothing or the like.

The mounting substrate in the housing 101 of the environmental sensor device 100 includes a rigid substrate 121 on which a CPU and an electronic circuit for processing measurement data, and a wireless communication chip for transmitting data to an external device are mounted, a sensor substrate on which the temperature and humidity sensor is mounted, a flexible substrate, and a battery for operating the temperature and humidity sensor 102. Of these parts, the rigid substrate 121 and the battery for operating the temperature and humidity sensor 102 are arranged in the sealed portion 104, and water, sweat, rain, or the like is prevented from entering from the outside.

The sealing structure of the sealed portion 104 may be constituted by a packing using an O ring or the like, and a screw or the like. If the housing 101 is formed of synthetic resin such as plastic, the sealing structure of the sealed portion 104 may be formed by means of welding using ultrasound or the like, or may be formed of an adhesive.

Meanwhile, the sensor substrate on which the temperature and humidity sensor 102 is mounted is arranged within the protective structure (unsealed portion) 105. The temperature and humidity sensor 102 is a sensor for measuring air temperature and relative humidity in the vicinity, is constituted by a semiconductor chip, for example, and includes a temperature sensor in which the resistance varies depending on temperature and a humidity sensor that absorbs moisture of surrounding gas and in which the capacity and resistance vary.

The temperature and humidity sensor 102 includes an AD conversion circuit, and the measured temperature and humidity are transmitted as digital data to the CPU in the sealed portion 104. The sensor substrate of the temperature and humidity sensor 102 is entirely protected by a chemically inactive coating (coating agent) to form a dustproof and waterproof structure. Note that the temperature and humidity sensor 102 itself is also dustproof and waterproof.

The temperature and humidity sensor 102 on the sensor substrate arranged in the protective structure (unsealed portion) 105 and the CPU on the rigid substrate 121 arranged in the sealed portion 104 are electrically connected to each other via the flexible substrate. The flexible substrate is arranged between the sealed portion 104 and the protective structure (unsealed portion) 105 such that the sealing property of the sealed portion 104 can be maintained by the packing using an O ring or the like, and the screw or the like, at the periphery of the housing wall 101C. In the arrangement of the flexible substrate at the housing wall 101C between the sealed portion 104 and the protective structure (unsealed portion) 105, for example, these parts may alternatively be adhered to each other without gaps using an adhesion, in addition to the aforementioned packing using an O ring or the like, and the screw or the like, and thus dustproof and waterproof properties can be ensured.

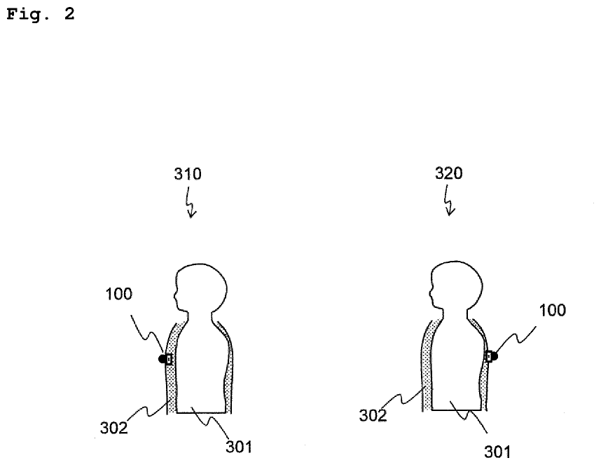

FIG. 2 shows examples in which the environmental sensor device 100 is attached to outerwear and thus worn and used. The outerwear 302 has a pocket, and the housing 101 of the environmental sensor device 100 is inserted into the pocket with the black bulb 1031 facing outward. For example, the environmental sensor device 100 may be worn at the chest position as in a wearing mode 310, or may be worn at the back position as in a wearing mode 320. If a pocket is provided, it is desirable that the material of the pocket is a highly moisture-permeable material such as a mesh fabric, in order to improve the response to humidity.

According to the above mode, it is possible to easily wear the environmental sensor device 100 at the same time as wearing clothing such as a T-shirt or a polo shirt, and measure environmental information such as temperature and humidity in the vicinity of a human body 301 without disturbing the wearer's movement.

It is also possible to easily remove the environmental sensor device 100 when measurement is not performed, and wash the clothing.

With the environmental sensor device 100 according to the present embodiment, the environment in the vicinity of a human body can be measured by measuring the influence of solar radiation on the human body using the black-bulb temperature sensor 103, and measuring temperature inside clothing and humidity inside clothing using the temperature and humidity sensor 102.

Although the present embodiment has described an example of measuring temperature and humidity inside clothing, the environmental sensor device can also be attached to the outside of outerwear and used as an external environmental sensor device attached to the immediate outside of a human body.

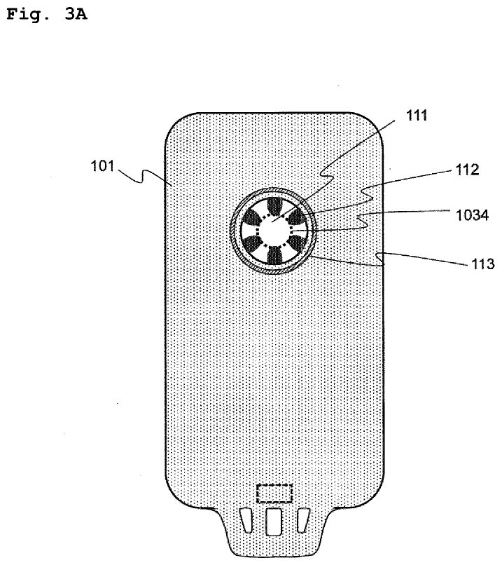

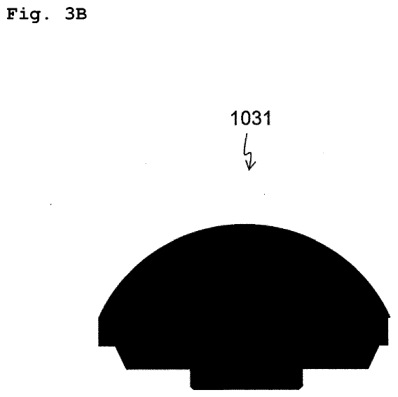

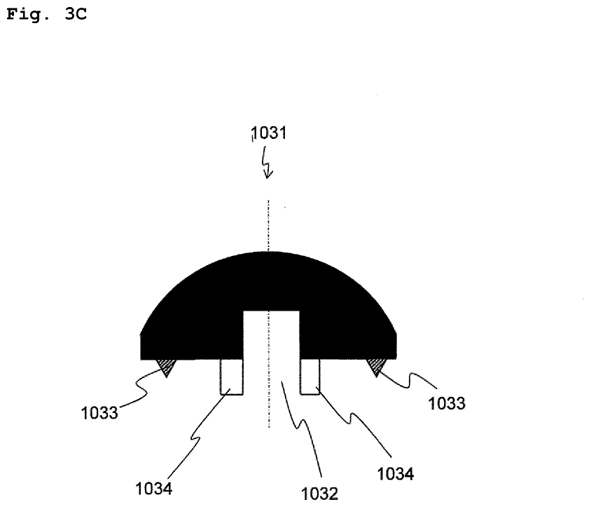

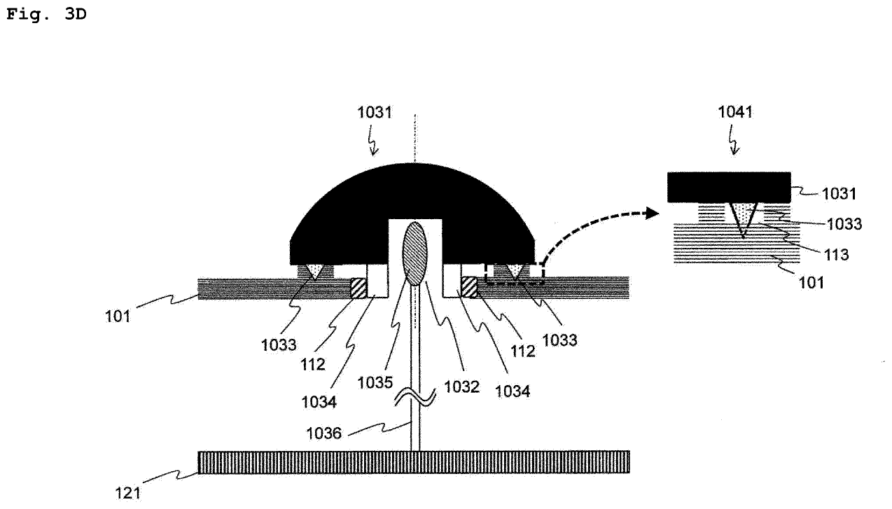

Next, joining between the housing 101 and the black bulb 1031 of the black-bulb temperature sensor 103 will be described with reference to FIGS. 3A to 3D. FIG. 3A shows the front face 101A of the housing 101 before the black bulb 1031 is joined thereto. FIG. 3B is a side view of the black bulb 1031. FIG. 3C is a cross-sectional view of the black bulb 1031 before the temperature sensor is arranged. FIG. 3D is a cross-sectional view of the black bulb 1031 after the temperature sensor is arranged.

The black bulb 1031 is formed of synthetic resin such as plastic for weight reduction, and it is desirable that the color of the black bulb 1031 is matte black and has an average emissivity of 0.95.

Joining of the black bulb 1031 to the housing 101 will be described. The black bulb 1031 has an insertion hole 1032 substantially at the center, a weld portion 1033 at an outer-circumferential portion of a bottom surface, and a guide portion 1034 at an outer-circumferential portion around the insertion hole 1032 (FIG. 3C). Meanwhile, the housing 101 has an insertion opening 111 into which the guide portion 1034 of the black bulb 1031 is inserted, and six protruding portions 112 extending inward from an outer-circumferential portion around the insertion opening 111. Further, the housing 101 has a weld region 113 at the outer circumferential portion around the insertion opening in the front face of the housing 101 (FIGS. 3A and 3B).

When the black bulb 1031 is welded to the housing 101, first, the guide portion 1034 of the black bulb 1031 is inserted into the insertion opening 111 of the housing 101. A dotted line in FIG. 3A indicates a portion into which the guide portion 1034 of the black bulb 1031 is inserted. At this time, the guide portion 1034 is supported by the protruding portions 112, and the weld portion 1033 abuts against the weld region 113.

Next, the weld portion 1033 of the black bulb 1031 is welded to the weld region 113 of the housing 101 using ultrasound or the like. An inserted diagram 1041 in FIG. 3D is an enlarged view of a region around the weld portion 1033 of the black bulb 1031 and the weld region 113 of the housing 101. A dotted line portion in the inserted diagram 1041 indicates a welded portion of a leading end of the weld portion 1033.

Black-bulb temperature is measured by inserting the temperature sensor 1035, such as a thermistor or a semiconductor temperature sensor, into the insertion hole 1032 in the black bulb 1031. A lead wire 1036 of the temperature sensor 1035 is electrically connected and fixed to the rigid substrate 121 arranged within the housing 101 by means of solder or the like. The measured temperature value may be corrected as required.

Thus, at the time of welding, a structure is formed in which the black bulb comes into contact with the housing 101 only at the guide portion 1034 of the black bulb and the protruding portions 112 of the housing 101, except for the weld portion 1033. Since the contact area between the black bulb 1031 and the housing 101 is reduced, heat conduction from the black bulb 1031 to the housing 101 during welding can be reduced. Further, a position shift during welding can be prevented by supporting the guide portion 1034 using the protruding portions 112.

As described above, the black bulb 1031 and the housing 101 are sealed by means of welding, and a dustproof and waterproof structure is formed. Accordingly, the environmental sensor device according to the present embodiment is appropriate for use in a high humidity environment and an environment where water droplets and moisture coming from sweat or the like are present.

Although the black bulb 1031 and the housing 101 are joined by means of welding in the present embodiment, they may be fixed to each other using an adhesive. However, when welding is used, a sufficient bonding strength can be achieved when only a portion of the outer-circumferential portion is used as the joint portion.

Here, if welding is used to join the black bulb 1031 to the housing 101, the shape and the diameter φ of the black bulb 1031 are also limited.

Although a size 1032 of the insertion opening in the black bulb 1031 is about 2 mm in diameter, it need only be a size that allows the temperature sensor to be inserted.

Further, it is desirable that the size of the black bulb 1031 is 9 mm or more in diameter considering that the guide portion 1034 is not affected by welding during the welding, and also considering the effect of reducing the contact area due to the protruding portions 112.

Meanwhile, when the wearable environment sensor device 100 is attached to clothing, it is difficult to attach if the spherical black bulb 1031 joined to the side face of the housing 101 is too large, and therefore a diameter of 24 mm or less is desirable considering the attachability.

If the surface area of the sphere is too small, the dependency on the attachment angle increases, resulting in a degradation of functions as a solar radiation sensor. On the other hand, if the black bulb is closer to a full sphere, the attachability including detachability deteriorates.

Therefore, it is desirable that the shape of the black bulb 1031 is a substantially hemispherical shape having a diameter of and 9 mm or more and 24 mm or less.

As described above, in the environmental sensor device 100 according to the present embodiment, the black bulb 1031 of the black-bulb temperature sensor 103 that needs to be in contact with outside air is arranged on an outer wall of the sealed portion (an outer wall of the sealed housing 101), and temperature inside the black bulb 1031 that increases due to the heat of solar radiation is detected by the temperature sensor 1035 arranged within the sealed portion 104 (the insertion hole 1032 in the black bulb 1031). A configuration is employed in which the rigid substrate 121 on which the CPU, electric circuit, and so on, for processing detected signals (digital data) are mounted is arranged together with the temperature sensor 1035 in the sealed portion 104 to ensure dustproof and waterproof properties. This configuration enables the temperature sensor 1035 to detect, with high sensitivity, temperature inside the black bulb 1031 that increases due to the heat of solar radiation, and enables detected information (digital data) to be stably processed by the CPU and the electronic circuit that are arranged in a sealed environment with excellent dustproof and waterproof properties.

The environmental sensor device 100 according to the present embodiment has a configuration in which the temperature and humidity sensor 102 that needs to be in contact with outside air is arranged within a portion outside the sealed portion (the protective structure (unsealed portion) 105), the rigid substrate 121 on which the CPU, the electronic circuit, and so on, for processing signals (digital data) obtained by the temperature and humidity sensor 102 are mounted is arranged in the sealed portion 104 to ensure dustproof and waterproof properties, and the temperature and humidity sensor 102 in the protective structure (unsealed portion) 105 is connected to the rigid substrate 121 in the sealed portion 104 by the flexible substrate. This configuration enables the temperature and humidity sensor 102 to come into contact with outside air and detect temperature and humidity with high sensitivity, in addition to the aforementioned detection of temperature by the black-bulb temperature sensor 103, and enables detected information (digital data) to be stably processed by the electronic circuit arranged in a sealed environment with excellent dustproof and waterproof properties.

Although the environmental sensor device 100 according to the present embodiment has the sealed portion with the black bulb provided on the outer wall and the protective structure (unsealed portion), a configuration only having the sealed portion with the black bulb provided on the outer wall also enables highly accurate and stable measurement using the black-bulb temperature sensor.

<Variations of First Embodiment>

Next, variations of the first embodiment will be described.

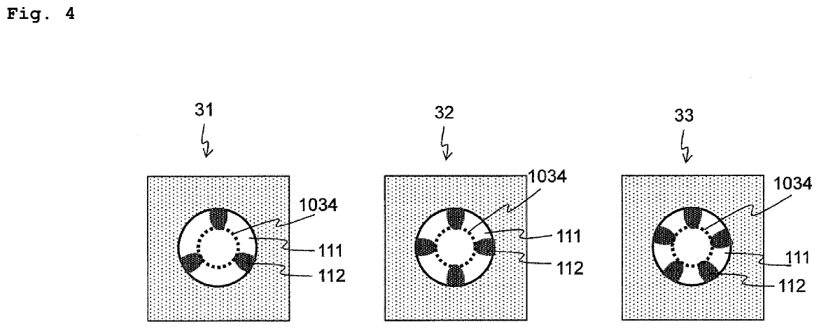

FIG. 4 shows the protruding portions 112 of the housing 101 in variations of the wearable environment sensor device according to the first embodiment. In the first embodiment, six protruding portions 112 are provided that come into contact with the guide portion 1034 of the black bulb 1031, as shown in FIG. 4. However, the number of protruding portions 112 may alternatively be three as indicated by 31 in FIG. 4, or may be four as indicated by 32 in FIG. 4, or may be five as indicated by 33 in FIG. 4.

Here, if the number of protruding portions 112 is two or less, the guide portion 1034 cannot be sufficiently fixed, and a position shift is more likely to occur during welding. If the number of protruding portions 112 is seven or more, the contact region increases, and thus the difference in thermal conductivity from when the protruding portions are not used and the outer wall of the guide portion 1034 comes into contact with an inner wall around the insertion opening over the entire region becomes 10% or less, degrading the thermal insulating effect. Accordingly, it is desirable that the number of protruding portions 112 that come into contact with the guide portion 1034 of the black bulb 1031 is three to six.

### Second Embodiment

Next, a wearable environment sensor device 200 according to the second embodiment will be described. The configuration of the wearable environment sensor device 200 according to the present embodiment is substantially the same as the configuration of the first embodiment, but is different in the shape of the guide portion.

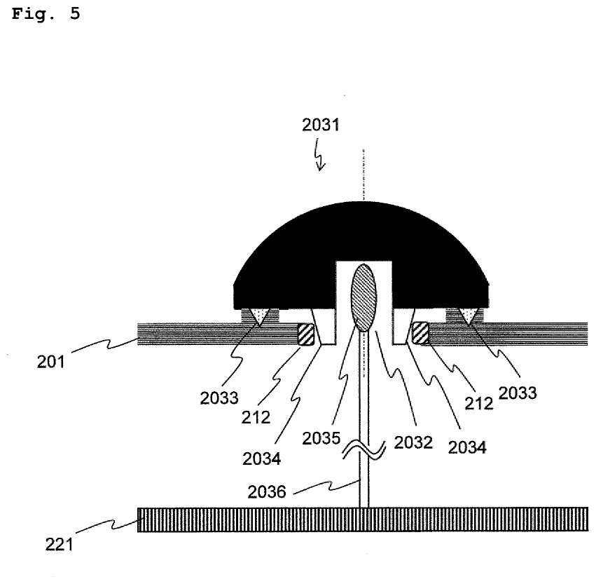

FIG. 5 is a cross-sectional view of a joint portion between the black-bulb temperature sensor and a housing 201 in the wearable environment sensor device 200 according to the second embodiment. The guide portion 1034 of the first embodiment has a cylindrical shape and comes into linear contact with the protruding portions 112. In contrast, a guide portion 2034 of the present embodiment employs a tapered structure in which the guide portion 2034 has a leading end tapered downward from the bottom surface of a black bulb 2031 (inward of the housing 201 when inserted into the housing 201), and therefore comes into point contact with protruding portions 212. As a result, the thermal conductivity can be reduced by 5% compared with the first embodiment.

As described above, according to the present embodiment, a further thermal insulating effect can be achieved in addition to the effects obtained in the first embodiment.

### INDUSTRIAL APPLICABILITY

Embodiments of the present invention relates to a wearable environment sensor device to be worn on a human body or the like, and is applicable to environmental measurement such as measurement of temperature and humidity in the vicinity of a human body, for example.

### REFERENCE SIGNS LIST

100 Wearable environment sensor device
101 Housing
103 Black-bulb temperature sensor
111 Insertion opening in housing
112 Protruding portion
1031 Black bulb
1032 Insertion hole in black bulb
1033 Weld portion
1034 Guide portion
1035 Temperature sensor
1036 Lead wire.

1-5. (canceled)

6. A wearable environment sensor device configured to measure environmental information regarding a place where the device is worn, the device comprising:
a black-bulb temperature sensor comprising a black bulb and a temperature sensor configured to measure an internal temperature in the black bulb, the black-bulb temperature sensor being provided in a housing;
wherein the black bulb comprises:
an insertion hole into which the temperature sensor is inserted, the insertion hole being provided in a bottom surface of the black bulb;
a weld portion welded to the housing in an outer-circumferential portion of the bottom surface; and
a guide portion in an outer-circumferential portion around the insertion hole; and
wherein the housing comprises:
an insertion opening into which the guide portion of the black bulb is inserted, wherein the weld portion of the black bulb is welded to the housing at an outer-circumferential portion around the insertion opening of the housing; and
a protruding portion at the outer-circumferential portion around the insertion opening, wherein the guide portion is supported by the protruding portion.

7. The wearable environment sensor device according to claim 6,
wherein a number of protruding portions of the housing is three or more; and
wherein the number of the protruding portions of the housing is six or less.

8. The wearable environment sensor device according to claim 6,
wherein the black bulb has a substantially hemispherical shape.

9. The wearable environment sensor device according to claim 8,
wherein the black bulb has a diameter of 9 mm or more; and
wherein the black bulb has the diameter of 24 mm or less.

10. The wearable environment sensor device according to claim 6,
wherein the guide portion has a tapered structure in which the guide portion is tapered inward of the housing from the bottom surface of the black bulb.

11. The wearable environment sensor device according to claim 6,
wherein the housing is sealed and has a dustproof structure.

12. The wearable environment sensor device according to claim 6,
wherein the housing is sealed and has a waterproof structure.

13. A wearable environment sensor device configured to measure environmental information, the device comprising:
a black-bulb temperature sensor comprising a black bulb and a temperature sensor configured to measure an internal temperature in the black bulb, the black-bulb temperature sensor being provided in and welded to a housing;
wherein the black bulb comprises:
an insertion hole at a bottom surface of the black bulb, wherein the temperature sensor is disposed in the insertion hole; and
a guide portion in an outer-circumferential portion around the insertion hole; and
wherein the housing comprises:
an insertion opening into which the guide portion of the black bulb is inserted, wherein the black bulb is welded to the housing around an outer-circumference of the insertion opening of the housing; and
a protruding portion supporting the guide portion.

14. The wearable environment sensor device according to claim 13, wherein the black bulb is welded to the housing around an outer-circumference of the bottom surface of the black bulb.

15. The wearable environment sensor device according to claim 13, wherein the protruding portion is disposed at the outer-circumference of the insertion opening of the housing.

16. The wearable environment sensor device according to claim 13, wherein the black bulb has a substantially hemispherical shape.
